(19) United States
(12) Patent Application Publication
(10) Pub. No.: US 2023/0226551 A1
(43) Pub. Date: Jul. 20, 2023

(54) SPECIMEN CUP HOLDER AND ILLUMINATION DEVICE

(71) Applicant: Specivue, LLC, Rockingham, VA (US)

(72) Inventor: Paul Guarino, Rockingham, VA (US)

(21) Appl. No.: 18/190,088

(22) Filed: Mar. 26, 2023

Related U.S. Application Data
(63) Continuation-in-part of application No. 17/162,389, filed on Jan. 29, 2021.

(51) Int. Cl. B01L 3/00

(52) U.S. Cl. CPC B01L 3/5085; B01L 2300/0809; B01L 2300/0851; B01L 2300/06

(57) ABSTRACT

A device includes a first container having a floor and walls extending from the floor to an opening opposing the floor. The walls include opposing ledges at an interior of the first container. A second container has a first open end, sides, and a second end opposing the first open end. The second end has at least one hole adapted to receive a biopsy specimen cup. The first and second containers are made from opaque materials. A translucent panel has a notch in an edge thereof. The translucent panel is disposed on the ledges and spans the interior of the first container. The opening of the first container is sized to slidingly receive the second container wherein the first open end of the second container rests on the translucent panel. A light source is disposed in the first container between the translucent panel and the floor of the first container.

This is a continuation-in-part application of co-pending application Ser. No. 17/162,389, “SPECIMEN CUP HOLDER AND ILLUMINATION DEVICE”, filed on Jan. 29, 2021.

### FIELD OF THE INVENTION

The invention relates generally to specimen cup holders, and more particularly to a device that holds multiple biopsy specimen cups while providing for illumination thereof such that biopsy specimens can be readily visualized in dark treatment room environments.

### BACKGROUND OF THE INVENTION

A variety of medical diagnoses are based on the analysis of human tissue specimens collected from internal regions of a patient. In many cases, the collected specimens are miniscule pieces of tissue collected using very small tools or instruments passed through an endoscope.

Medical procedures that utilize endoscopes are generally conducted in dark rooms so that attending medical personnel can clearly see images transmitted by the endoscope to a display being closely monitored by medical personnel carrying out the procedure. Each collected specimen is passed to an attending nurse or technician who must then place the specimen in a specimen cup. Combining miniscule specimen collection in a dark room with the desire to minimize the length of an endoscopic procedure frequently results in specimen collection issues to include loss of specimens and the inability to verify if a specimen is actually in a specimen cup.

### SUMMARY OF THE INVENTION

Accordingly, it is an object of the present invention to provide a device that aids in the human tissue specimen collection process that takes place during an endoscopic procedure.

Another object of the present invention is to provide a device that can be used in a dark endoscopic-procedure room to improve the process of human tissue specimen collection and verification.

Other objects and advantages of the present invention will become more obvious hereinafter in the specification and drawings.

In accordance with the present invention, a device includes a first container having a floor and walls extending from the floor to an opening in the first container. The opening opposes the floor. The walls include opposing ledges at an interior of the first container. A second container has a first open end, sides, and a second end opposing the first open end. The second end has at least one hole adapted to receive a biopsy specimen cup up to the specimen cup's outer annular flange. The first container and second container are made from opaque materials. A translucent panel has a notch in an edge thereof. The translucent panel is disposed on the ledges and spans the interior of the first container wherein the notch is adjacent to one of the walls of the first container and wherein the translucent panel is spaced apart from the floor of the first container. The opening of the first container is sized to slidingly receive the second container wherein the first open end of the second container rests on the translucent panel. A light source is disposed in the first container between the translucent panel and the floor of the first container.

### BRIEF DESCRIPTION OF THE DRAWINGS

Other objects, features and advantages of the present invention will become apparent upon reference to the following description of the preferred embodiments and to the drawings, wherein corresponding reference characters indicate corresponding parts throughout the several views of the drawings and wherein:

FIG. 1 is a perspective view of a specimen cup holder and illumination device in accordance with an embodiment of the present invention;

FIG. 2 is a cross-sectional view of the device taken along 2-2 in FIG. 1;

FIG. 3 is a top plan view of the device illustrated in FIG. 1 with its opaque top omitted therefrom;

FIG. 4 is an isolated longitudinal cross-sectional view of the device's top container illustrated in FIG. 1;

FIG. 5 is a cross-sectional view of the device's bottom container illustrated in FIG. 1;

FIG. 6 is an exploded perspective view of the device's top container having a removable top in accordance with another embodiment of the present invention;

FIG. 7 is an isolated perspective view of the top container of a specimen cup holder and illumination device having a non-illuminated specimen-manipulating tool holder in accordance with another embodiment of the present invention;

FIG. 8 is a longitudinal cross-sectional view of the top container illustrated in FIG. 7;

FIG. 9 is a cross-sectional view of the device's bottom container in accordance with another embodiment of the present invention;

FIG. 10 is an isolated plan view of the ledge-forming removable end wall of the bottom container illustrated in FIG. 9;

FIG. 11 is an exploded perspective view of a specimen cup holder and illumination device in accordance with another embodiment of the present invention;

FIG. 12 is an isolated top plan view of the device's bottom container taken along 12-12 in FIG. 11;

FIG. 13 is an isolated top plan view of the device's bottom container with the device's translucent panel installed therein taken along 13-13 in FIG. 11;

FIG. 14 is an isolated bottom plan view of the device's top container taken along 14-14 in FIG. 11;

FIG. 15 is a top plan view of the assembled device taken along line 15-15 in FIG. 11;

FIG. 16 is a cross-sectional view of an assembled device taken along line 16-16 in FIG. 15; and

FIG. 17 is a cross-sectional view of an assembled specimen cup holder and illumination device in accordance with another embodiment of the present invention.

### DETAILED DESCRIPTION OF THE INVENTION

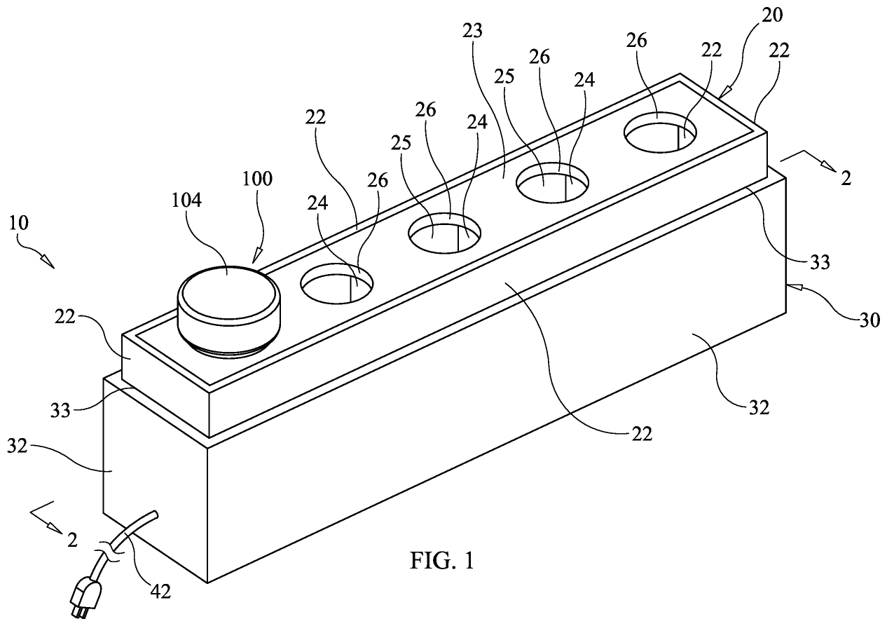

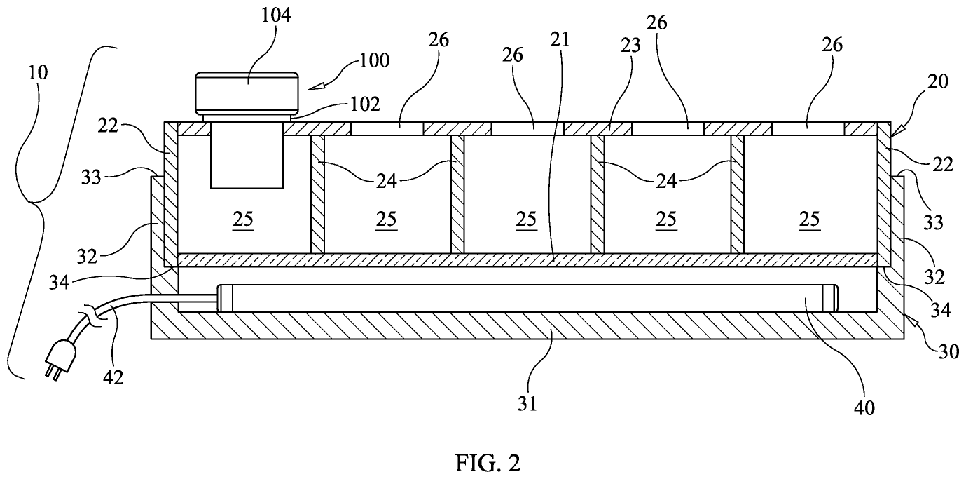

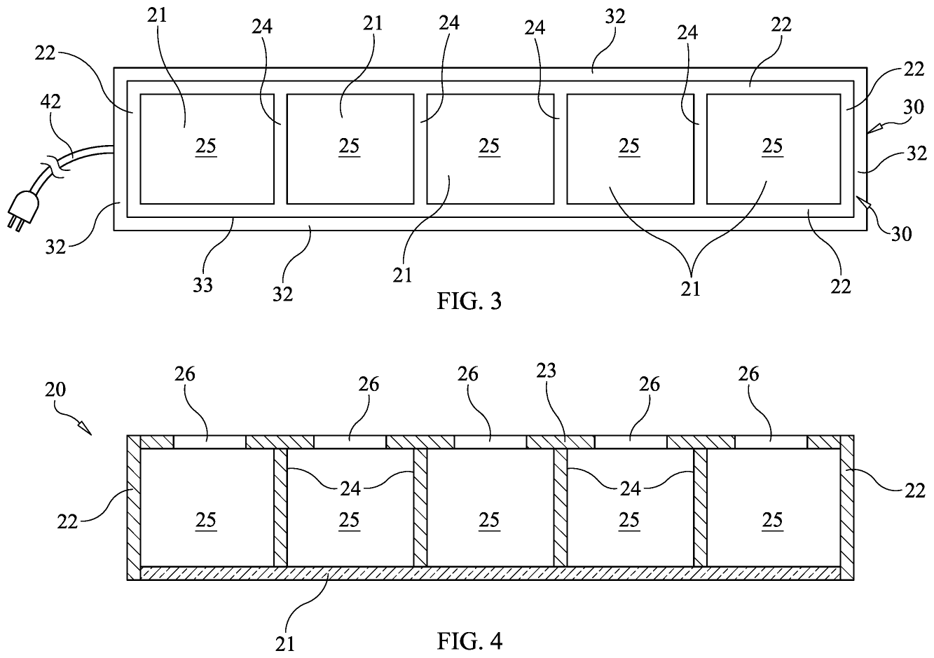

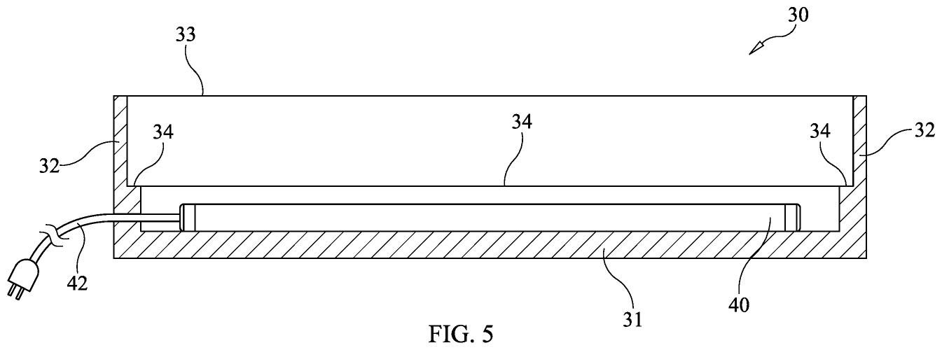

Referring now to the drawings, simultaneous reference will be made to FIGS. 1-5 where a specimen cup holder and illumination device is illustrated in its entirety in FIGS. 1-2 and in portions thereof in FIGS. 3-5. The entire device is referenced generally by numeral 10 in FIGS. 1-2. As will be explained further herein, device 10 simultaneously holds and illuminates multiple specimen cups in which human tissue specimens are placed during an endoscopic procedure. In FIGS. 1 and 2, a typical specimen cup 100 is shown being supported in device 10.

Device 10 includes two containers 20 and 30, and a light source 40. Briefly, container 20 (also referred to herein as the “top container”) holds/supports one or more specimen cups (e.g., specimen cup 100), and container 30 (also referred to herein as the “bottom container”) holds/supports top container 20 and light source 40. As will be explained further below, when light source 40 is turned on to generate light, any specimen cup held in top container 20 is back-lit with diffuse light.

Top container 20 is a generally rigid container made from one or more materials suitable for use in a medical setting where such materials are readily cleaned and disinfected in accordance with established guidelines and procedures. In the illustrated embodiment, top container 20 is generally rectangular in shape. However, it is to be understood that top container 20 could be realized by other geometric shapes without departing from the scope of the present invention.

Top container 20 is constructed to have a translucent bottom 21, opaque sides 22, and an opaque top 23. Top 23 can be fixed in place or can be removable from top container 20 as will be explained further below. Disposed within top container 20 are a number of interior walls 24 (e.g., four in the illustrated embodiment). Each interior wall 24 is opaque and spans between translucent bottom 21 and opaque top 23 as well as between opposing ones of the top container's long sides 22. As a result, a number of optically isolated compartments 25 are defined within top container 20 where each compartment 25 includes a portion of translucent bottom 21.

Opaque top 23 has a number of through holes 26 with each hole 26 positioned to communicate with one of compartments 25. Each hole 26 is sized/shaped to receive a specimen cup therein. Since specimen cups, such as specimen cup 100, generally have an outer annular flange 102 to provide for sealing engagement with the cup's top 104, holes 26 are sized/shaped such that flange 102 rests on top 23 as illustrated. Top container 20 can be sized such that the bottom of specimen cup 100 is suspended above translucent bottom 21 as flange rests on top 23.

Bottom container 30 is also a generally rigid container made from one or more materials suitable for use in a medical setting for purposes of cleaning, disinfecting, etc. The shape/size of bottom container 30 is predicated on that of top container 20. In general, bottom container 30 is an open-top container configured to slidingly receive a portion of top container 20 therein to include its translucent bottom 21 such that translucent bottom is spaced from the closed bottom of bottom container 30. More specifically, bottom container 30 has a closed opaque bottom 31 and opaque sides 32 with its open top 33 sized/shaped to slidingly receive a portion of top container 20 therein. Bottom container 30 also has an interior annular ledge 34 on which top container 20 rests so that translucent bottom 21 is suspended above closed bottom 31.

Light source 40 is disposed in bottom container 30 in the region thereof beneath translucent bottom 21. In the illustrated embodiment, light source 40 is an elongated light source that generates light substantially all along the length of translucent bottom 21 such that light is generated beneath each of compartments 25. The generated light is diffused by translucent bottom 21 before it illuminates each of compartments 25. Light source 40 can be powered via an electric power cord 42 led into bottom container 30 as illustrated. However, the present invention is not so limited as light source 40 could also be battery-powered without departing from the scope of the present invention. In general, minimal or no heat should be generated by light source 40 when turned on to prevent heat build-up within device 10 that could negatively impact the tissue specimens being collected. Accordingly, suitable lights for light source 40 include fluorescent lights, LED lights, or any light source that generates minimal or no heat.

In use, one or more specimen cups are placed in holes 26 and light source 40 is turned on. The light produced by source 40 is diffused by translucent bottom 21 so that the specimen cups are back lit with diffuse light. In this way, when a specimen cup's top is removed, the contents of the specimen cup are evenly illuminated for viewing by a technician. At the same time, the generated light is predominantly contained within device 10 thereby preventing the light from negatively impacting the surrounding procedure environment and personnel.

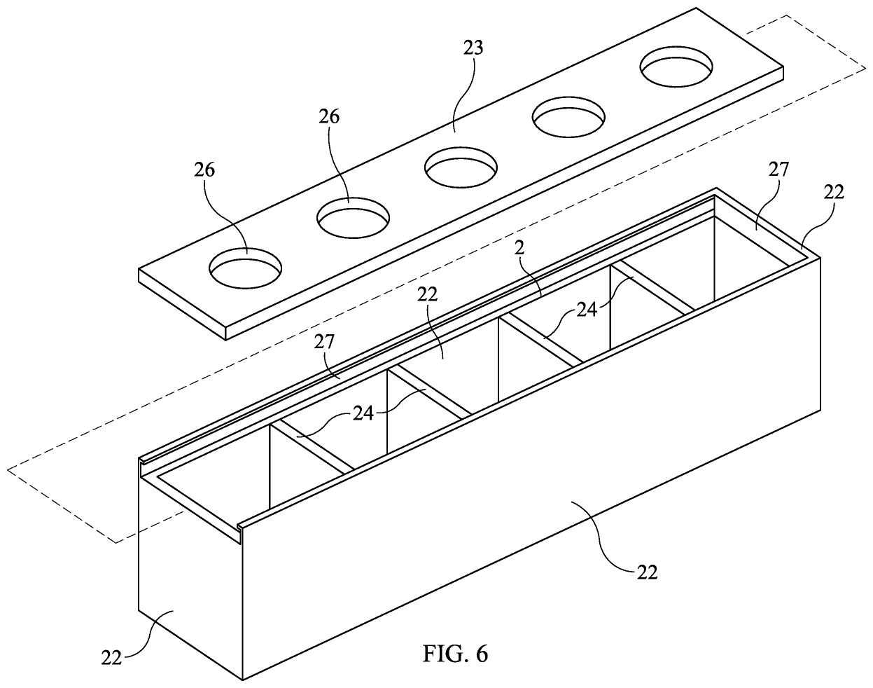

As mentioned above, the opaque top 23 of top container 20 can be made removable to simplify the cleaning/disinfecting thereof, to provide for ease of its replacement, and/or provide for tops 23 having holes 26 of varying size to accommodate different sizes of specimen cups. For example and as illustrated in FIG. 6, an open-ended slot 27 can be formed in the top regions of three of opaque sides 22 such that opaque top 23 can be readily slid into and out of slot 27. When fully inserted in slot 27, top 23 has the same attributes described previously herein where each hole 26 is aligned with one of compartments 25. It is to be understood that top 23 can be made removable in other ways without departing from the scope of the present invention.

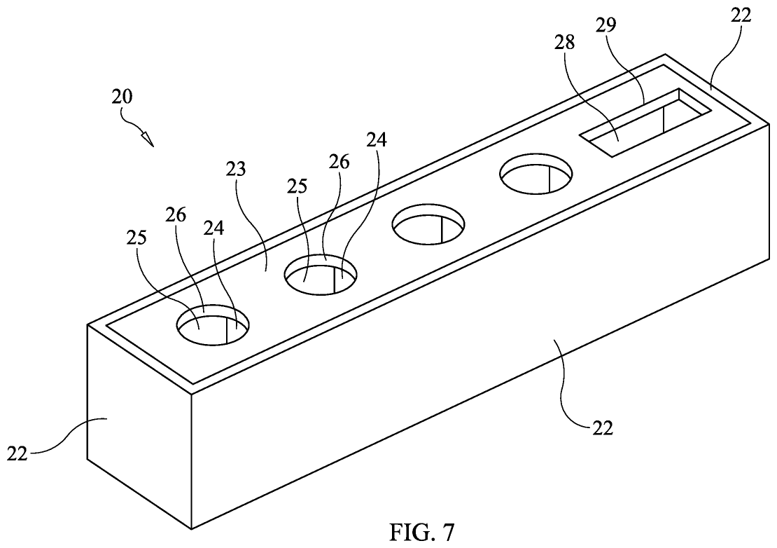

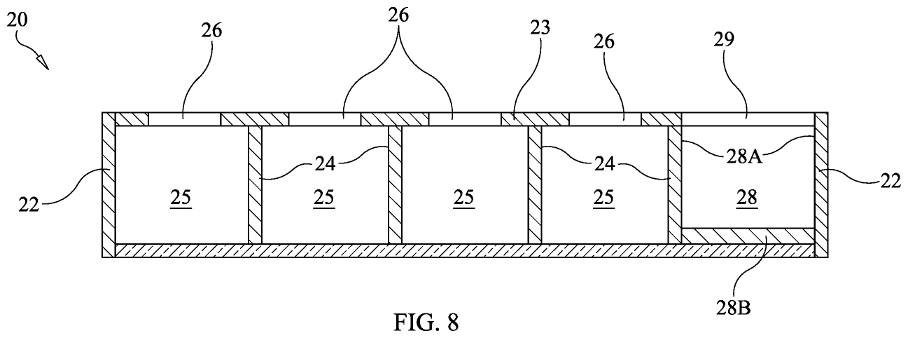

Another embodiment of the top container of the present invention is illustrated in FIGS. 7-8 wherein common reference numerals are used for those elements previously described herein. In this embodiment of top container 20, an additional compartment 28 is provided in top container 20 to provide a place to store/hold one or more specimen-manipulating tools commonly used by a technician tasked with populating specimen cups during an endoscopic procedure. More specifically, compartment 28 is accessed via a hole 29 in opaque top 23. Compartment 28 is constructed in a way that keeps it optically isolated from translucent bottom 21 as well as any adjacent compartment 25 in order to prevent light from entering and escaping from compartment 28 via hole 29. For example, in the illustrated embodiment, compartment 28 is defined by opaque sides 28A (i.e., sides 28A can be shared with or realized by one or more of opaque sides 22 and interior walls 24) and an opaque bottom 28B that prevent light from being passed into compartment 28 via translucent bottom 21. The shape/size of compartment 28 and its access hole 29, as well as how it is constructed for optical isolation, are not limitations of the present invention.

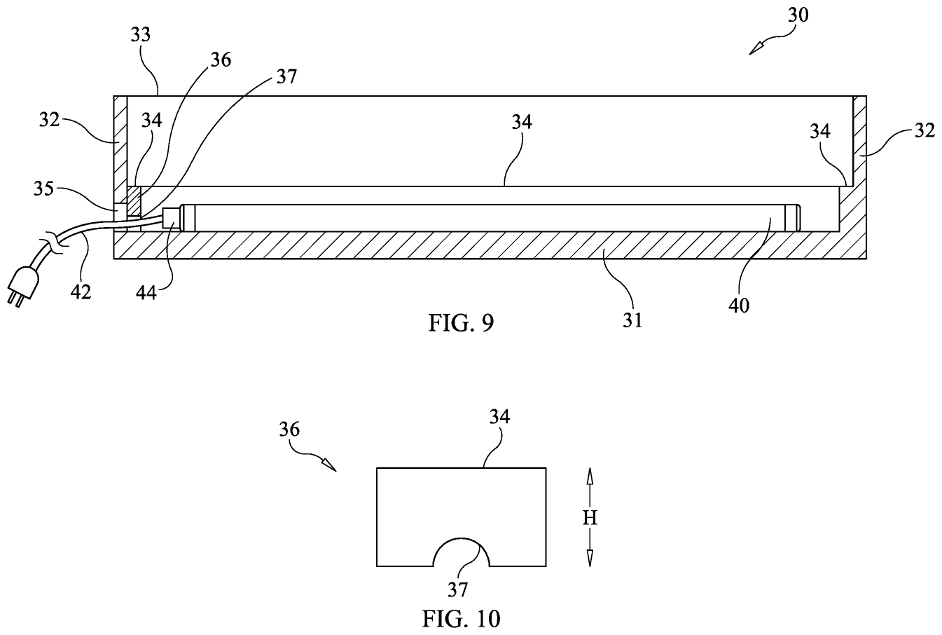

In another embodiment illustrated in FIGS. 9 and 10, bottom container 30 and light source 40 (FIG. 9) provide for the removal of light source 40 to facilitate a complete cleaning of bottom container 30. More specifically, light source 40 has its power cord 42 terminating in removable plug 44 attachable to light source 40. Since plug 44 will generally be larger in diameter than power cord 42, a hole 35 in side 32 must be large enough to allow plug 44 to pass there through. Unfortunately, a hole 35 sized in this fashion will also allow light from light source 40 to escape from bottom container 30 which can serve as a distraction during a medical procedure as described above. To prevent this, a removable opaque end wall 36 (shown in isolation in FIG. 10) is notched or is cut out at 37 to snugly fit over power cord 42 just before it passes through hole 35. That is, notch/cutout 37 has a smaller size/diameter than that of hole 35 so that minimal light will escape through hole 35 when light source 40 is illuminated. The height “H” of end wall 36 is such that the top thereof aligns with and becomes part of annular ledge 34 when end wall 36 abuts a side 32 and faces into bottom container 30 as illustrated.

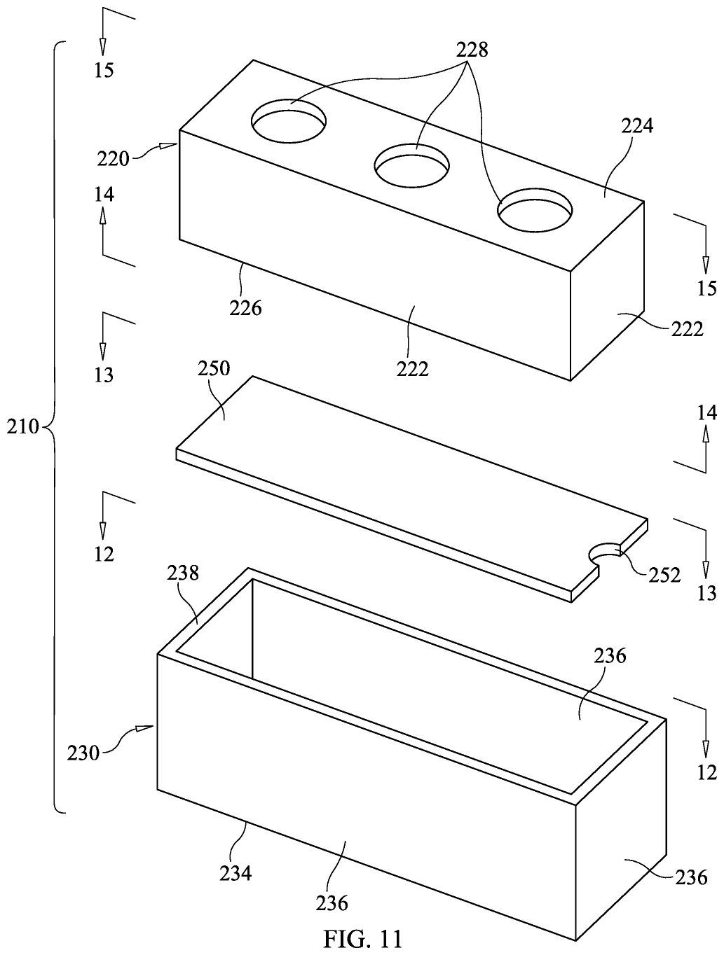

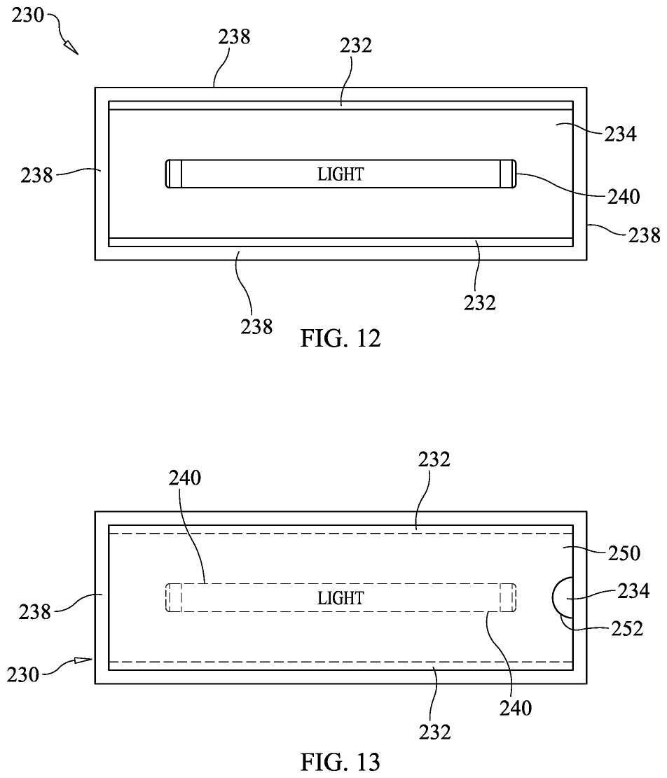

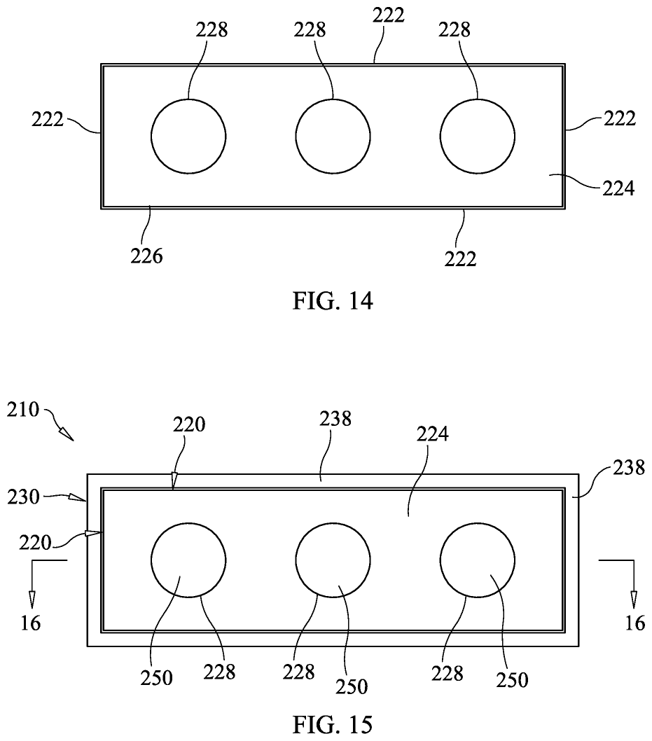

Another embodiment of a specimen cup holder and illumination device is illustrated in FIGS. 11-16. Simultaneous reference will be made to FIGS. 11-16 where the specimen cup holder and illumination device 210 is illustrated in an exploded view in FIG. 11 and in various isolated views and stages of assembly in FIGS. 12-16. Similar to the embodiments described previously herein, device 210 simultaneously holds/supports and illuminates one or more specimen cups (e.g., specimen cup 100 shown in FIGS. 1 and 2) in which human tissue specimens are placed during an endoscopic procedure. For clarity of illustration, no specimen cup is shown with device 210.

Device 210 includes two containers 220 and 230, a light source 240 (not visible in FIG. 11), and a translucent panel 250. Briefly, container 220 (also referred to herein as the “top container”) holds/supports one or more specimen cups (not shown), and container 230 (also referred to herein as the “bottom container”) holds/supports translucent panel 250, top container 220, and light source 240. As will be explained further below, when light source 240 is turned on to generate light, the generated light is diffused by translucent panel 250 prior to back-lighting any specimen cup held in top container 220.

Top container 220 is a generally rigid container made from one or more materials suitable for use in a medical setting where such materials are readily cleaned and disinfected in accordance with established guidelines and procedures. In the illustrated embodiment, top container 220 is generally rectangular in shape. However, it is to be understood that top container 220 could be realized by other geometric shapes without departing from the scope of the present invention.

Top container 220 is constructed from opaque material(s) to have sides 222, a top 224 coupled to sides 222, and an open bottom 226 that is in opposition to top 224. Top 224 has one or more through holes 228 (e.g., three in the illustrated embodiment). As described earlier herein, each hole 228 is sized/shaped to receive a specimen cup therein and support it via the specimen cup's outer annular flange (e.g., flange 102 shown in FIG. 2). Top container 220 may be sized such that the bottom of a specimen cup is suspended above translucent panel 250 as will be explained further below.

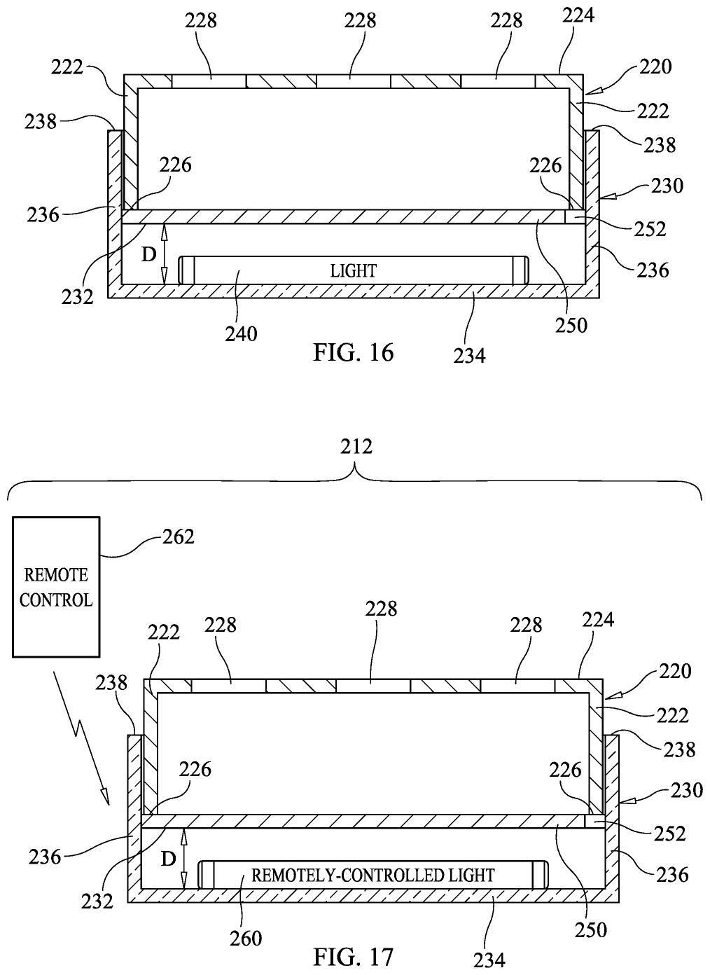

Bottom container 230 is also a generally rigid container made from one or more opaque materials suitable for use in a medical setting for purposes of cleaning, disinfecting, etc. The shape/size of bottom container 230 is predicated on that of top container 220, i.e., rectangular in the illustrated example. In general, bottom container 230 is an open-top container configured to slidingly receive a portion of top container 220 therein where the amount of such contained portion of top container 220 is determined by two ledges 232 (not visible in FIG. 11) provided on the interior of bottom container 230. Ledges 232 can be coupled to or integrated with bottom container 230. In the illustrated embodiment, ledges 232 extend all long two opposing long sides of bottom container 230 and at the same height “D” relative to floor 234 (FIG. 16). Bottom container 230 has a closed bottom or floor 234, walls 236 coupled to or integrated with floor 234 and extending up to the container's open top 238 that is in opposition to floor 234. Open top 238 and the interior of bottom container 230 up to ledges 232 is sized/shaped to slidingly receive a portion of top container 220 therein in a close fit relationship, e.g., sides 222 and the interior of walls 236 may be in sliding contact with one another.

Light source 240 is disposed in bottom container 230 in the region thereof beneath ledges 232. Light source 240 may rest on floor 234 and may be removably coupled to floor 234 in ways well-understood in the art. Light source 240 may be of a length to substantially extend along the length of bottom container 230 to thereby generate light substantially all along the length of bottom container 230. Light source 240 may be a battery-powered (e.g., by a rechargeable battery). In general, minimal or no heat should be generated by light source 240 when turned on to prevent heat build-up within device 210 that could negatively impact the tissue specimens being collected. Accordingly, suitable lights for light source 240 include fluorescent lights, LED lights, or any light source that generates minimal or no heat.

Translucent panel 250 is a single piece of semi-rigid or rigid translucent material (e.g., white plexiglass or acrylic) that is disposed in bottom container 230 and is sized/shaped to rest on ledges 232 while spanning the interior of bottom container 230. A notch 252 is provided in an edge of panel 250. In general, notch 252 is sized/shaped to facilitate the lifting of panel 252 from ledges 232. In the illustrated embodiment, notch 252 is located on panel 250 such that notch 252 is adjacent to one of the bottom container's walls 236 that does not include a ledge 232 such that there is no ledge to impede the lifting of panel 250 via notch 252.

In use, panel 250 is placed in bottom container 230 to rest on ledge 232. Top container 220 is then placed in bottom container 230 where the top container's open bottom 226 will rest on panel 250 while extending partially out of open top 238 as illustrated in FIG. 16. One or more specimen cups are placed in holes 228 and light source 240 is turned on. The light produced by source 240 is diffused by translucent panel 250 so that any specimen cup disposed in a hole 228 is back lit with diffuse light. In this way, when a specimen cup's top is removed, the contents of the specimen cup are evenly illuminated for viewing by a technician. At the same time, the generated light is predominantly contained within device 210 thereby preventing the light from negatively impacting the surrounding procedure environment and personnel.

Another embodiment of a specimen cup holder and illumination device 212 is illustrated the cross-section view presented in FIG. 17. Device 212 is similar to the above-described 210 except that the light source disposed in bottom container 230 is a remotely-controlled light source 260 that may be operably controlled (e.g., on/off, light level, light color, etc.) by a remote control 262.

The advantages of the present invention are numerous. The specimen cup holder and illumination device allows multiple specimen cups to be simultaneously supported, while allowing a single cup whose top is removed to be illuminated with diffuse light so that miniscule tissue specimens can be readily placed and verified during an endoscopic procedure. By trapping nearly all light within the device, the preferred dark room environment of an endoscopic procedure can be maintained thereby ensuring the efficacy and efficiency of the procedure. Since a specimen-handling technician can readily verify specimen collection, an endoscopic procedure can proceed quickly to minimize patient trauma, duration of patient sedation, and the utilization time of a facility's endoscopy/operating room. The device is easily disassembled (for cleaning) and reassembled without the need for any tools.

Although the invention has been described relative to specific embodiments thereof, there are numerous variations and modifications that will be readily apparent to those skilled in the art in light of the above teachings. It is therefore to be understood that, within the scope of the appended claims, the invention may be practiced other than as specifically described.

What is claimed as new and desired to be secured by Letters Patent of the United States is:

1. A device, comprising:
a first container having a floor and walls extending from said floor to an opening in said first container, said opening opposing said floor, said walls including opposing ledges at an interior of said first container;
a second container having a first open end, sides, and a second end opposing said first open end, said second end having at least one hole adapted to receive a biopsy specimen cup up to the specimen cup's outer annular flange;
said first container and said second container made from opaque materials;
a translucent panel having a notch in an edge of said translucent panel, said translucent panel disposed on said ledges and spanning said interior of said first container wherein said notch is adjacent to one of said walls of said first container and wherein said translucent panel is spaced apart from said floor of said first container;
said opening of said first container sized to slidingly receive said second container wherein said first open end of said second container rests on said translucent panel; and
a light source disposed in said first container between said translucent panel and said floor of said first container.

2. The device of claim 1, wherein said first container and said second container are rectangular.

3. The device of claim 1, wherein said light source is remotely controllable, said device further comprising a remote control operable to control said light source.

4. The device of claim 1, wherein said light source rests on said floor.

5. The device of claim 1, wherein a portion of said second container extends from said opening of said first container when said second container rests on said translucent panel.

6. A device, comprising:
a first rectangular container having a floor and four walls extending from said floor to an open top of said first rectangular container, said open top opposing said floor;
a first wall of said four walls having a first ledge at an interior of said first rectangular container, said first ledge spaced apart from said floor;
a second wall of said four walls in opposition to said first wall, said second wall having a second ledge at said interior of said first rectangular container, said second ledge spaced apart from said floor;
a second rectangular container having a first open end, sides, and a second end opposing said first open end, said second end having at least one hole adapted to receive a biopsy specimen cup up to the specimen cup's outer annular flange;
said first rectangular container and said second rectangular container made from opaque materials;
a translucent panel having a notch in an edge of said translucent panel, said translucent panel resting on said first ledge and said second ledge, said translucent panel spanning said four walls at said interior of said first container, wherein said notch is adjacent to a third wall of said four walls or a fourth wall of said four walls, and wherein said translucent panel is spaced apart from said floor of said first container;
said opening of said first container sized to slidingly receive said second container wherein said first open end of said second container rests on said translucent panel; and
a light source disposed in said first container between said translucent panel and said floor.

7. The device of claim 6, wherein said light source is remotely controllable, said device further comprising a remote control operable to control said light source.

8. The device of claim 6, wherein said light source rests on said floor.

9. The device of claim 6, wherein a portion of said second rectangular container extends from said opening of said first rectangular container when said first open end of said second rectangular container rests on said translucent panel.

10. The device of claim 6, wherein said first ledge and said second ledge are equidistant from said floor.

11. The device of claim 6, wherein a bottom of the specimen cup is suspended above said translucent panel when said first open end of said second rectangular container rests on said translucent panel.

12. A device, comprising:
a first rectangular container having a floor and four walls extending from said floor to an open top of said first rectangular container, said open top opposing said floor;
a first wall of said four walls having a first ledge at an interior of said first rectangular container, said first ledge extending along said first wall at a distance from said floor;
a second wall of said four walls in opposition to said first wall, said second wall having a second ledge at said interior of said first rectangular container, said second ledge extending along said second wall at said distance from said floor;
a second rectangular container having a first open end, sides, and a second end opposing said first open end, said second end having at least one hole adapted to receive a biopsy specimen cup up to the specimen cup's outer annular flange;
said first rectangular container and said second rectangular container made from opaque materials;
a translucent panel having a notch in an edge of said translucent panel, said translucent panel resting on said first ledge and said second ledge, said translucent panel spanning said four walls at said interior of said first container, wherein said notch is adjacent to a third wall of said four walls or a fourth wall of said four walls, and wherein said translucent panel is spaced apart from said floor of said first container;
said opening of said first rectangular container sized to slidingly receive said second rectangular container, wherein said first open end of said second rectangular container rests on said translucent panel, wherein a portion of said second rectangular container extends from said opening of said first rectangular container when said first open end of said second rectangular container rests on said translucent panel, and wherein a bottom of the specimen cup is suspended above said translucent panel when said first open end of said second rectangular container rests on said translucent panel;
a remotely-controllable light source disposed on said floor of said first rectangular container and spaced apart from said translucent panel; and
a remote control operable to control said light source.
